(19) United States
(12) Patent Application Publication
(10) Pub. No.: US 2025/0295403 A1
(43) Pub. Date: Sep. 25, 2025

(54) Systems, Methods, and Apparatuses for Delivering Bone Anchors

(71) Applicant: Marc F. Matarazzo, Longview, TX (US)

(72) Inventor: Marc F. Matarazzo, Longview, TX (US)

(21) Appl. No.: 18/610,722

(22) Filed: Mar. 20, 2024

(51) Int. Cl. A61B 17/04; A61B 17/00

(52) U.S. Cl. CPC A61B 17/0401; A61B 2017/00367; A61B 2017/00477; A61B 2017/00836; A61B 2017/00862; A61B 2017/00867; A61B 2017/00946; A61B 2017/0409

(57) ABSTRACT

A bone anchor inserter for delivering bone anchors linked by a suture with a slidingly, locking knot through the soft tissue of a rotate cuff tendon into the humerus is provided. The bone anchor inserter includes a body having a distal end coupled to a near end of a cylindrical tube. A solid cylindrical rod is slidingly located within the cylindrical tube. The distal end of the solid cylindrical rod is terminated with a two prong fork configured support the pair of bone anchors prior to development. A top plate is coupled to a near end of the bone inserter body includes an aperture to allow the tail of the suture to pass through. A grip configured to position and manipulate the distal end of the cylindrical tube is located on the underside of the body of the bone anchor behind a trigger configured to deploy the pair of bone anchors.

### FIELD

The present disclosure relates to an apparatus for delivering bone anchors during a medical procedure through the soft tissue of a rotator cuff tendon to the underlying bone of the upper portion of the arm bone or humerus, specifically the apparatus is a hand-held medical bone anchor inserter that provides delivery of soft tissue fixation to bone at two anchor points, while using sutures and a pre-loaded sliding knot to apply quick and easy compression along the soft tissue-bone interface between the two anchor points.

### BACKGROUND OF THE INVENTION

Tears either partial or complete in the soft tissue of the rotator cuff tendon have long been recognized as a source of pain and weakness in the shoulder. Tearing of the rotator cuff can occur when these tendons become irritated and swollen, and eventually wear out, or they can occur as a result of direct injury. Most tears are preceded by at least some degree of tendon wear damage. Any accidents or injuries that might occur at work, sport or a fall may precipitate a tear of these weakened tendons. Those patients with large rotator cuff tears have difficulty raising their arm or rotating the arm out towards the side. Tears of the rotator cuff become increasingly common with age. While it is less common for patients who are younger than 40 years old to have a rotator cuff tear it is not uncommon for patients who are older than 70 years old to have at least one rotator cuff tear. There are a number of surgical procedures to address a rotator cuff tear, including anchoring an initial bone-soft tissue implant to an independently delivered second bone-only implant with a suture under tension. However, the medical procedure requires the surgeon to fixate the second implant to the initial implant with sutures under tension using complicated locking knots during the actual medical procedure.

What is needed is a hand-held medical bone anchor inserter that allows delivery of soft tissue fixation to bone at two points of fixation using a pair of pre-load bone anchors linked by at least one suture comprising a sliding and locking knot to enable quick and easy compression to be applied along the soft tissue-bone interface between the two points of fixation. The proposed hand-held medical bone anchor inserter allows compressive fixation between two points of soft tissue-bone anchors utilizing a pre-made, quick, and easy sliding locking suture knot.

### SUMMARY OF THE INVENTION

The present disclosure relates to an apparatus for delivering bone anchors during a medical procedure through the soft tissue of a tendon to the underlying bone. Specifically, the apparatus is a hand-held medical orthopedic implant device that allows delivery of soft tissue fixation to bone at two anchor points, using a pair of bone anchors coupled by a suture having a sliding locking knot. This configuration is used to apply quick and easy compression along the soft tissue-bone interface between the two anchor points, such that the soft tissue of a tendon is secured to the upper portion of the arm bone or humerus during a surgical procedure.

In various embodiments, a bone anchor inserter includes a longitudinal body and a cylindrical tube having a near end coupled to a distal end of the longitudinal body. The bone anchor inserter also includes a solid cylindrical rod slidingly located within the cylindrical tube. The distal end of the solid cylindrical rod is configured to retain a first bone anchor and a second bone anchor. The bone anchor inserter also includes a top plate coupled to a near end of the longitudinal body and a deployment trigger coupled to a middle of the underside of the longitudinal body. The deployment trigger is configured to deploy the first bone anchor and the second bone anchor retained by the distal end of the solid cylindrical rod. The bone anchor inserter also includes a grip coupled to a rear of the underside longitudinal body. The grip is operable to position and manipulate the distal end of the cylindrical tube.

In various embodiments, the cylindrical tube is pre-loaded with the first bone anchor and the second bone anchor. The first bone anchor and the second bone anchor are coupled by a suture and a sliding, locking knot.

In various embodiments, the suture and the sliding knot are configured to apply a compression force along a soft-tissue bone interface between the first bone anchor and the second bone anchor.

In various embodiments, the top plate includes an eyelet aperture formed by a longitudinal cavity in the center of the top plate. The longitudinal cavity in the center of top plate is configured to allow at least a tail of the suture to be fed through the eyelet aperture

In various embodiments, the distal end of the cylindrical tube is in fluid communication with the eyelet aperture formed by the longitudinal cavity in the center of the top plate.

In various embodiments, the distal end of the solid cylindrical rod is coupled to and terminated with a two prong fork. The two prong fork is configured to straddle the first bone anchor.

In various embodiments, the near end of the cylindrical tube is operable to lockingly engage the distal end of the longitudinal body. In some embodiments, the near end of the cylindrical tube is threaded and configured to engage a matching threaded recess in the distal end of the longitudinal body.

In various embodiments, the length of the cylindrical tube is between about 15 cm and about 25 cm. For example, the length of the cylindrical tube is about 22 cm.

In various embodiments, the distal end of the cylindrical tube tapers to a sharp point configured to make a pilot hole in bone for a bone anchor. In some embodiments, the length of the sharp point is between about 10 mm and about 20 mm. For example, the length of the sharp point is about 15 mm.

In various embodiments, one or more of the solid cylindrical rod and the cylindrical tube is made of a metal, or metal-like material. In some embodiments, the device is disposable wherein the gun section may be re-usable with disposable cartridges housing the anchors and suture.

In various embodiments, the distal end of the cylindrical tube includes a retractable delivery chute.

In various embodiments, the length of the solid cylindrical rod is between about 15 cm and about 25 cm and the diameter the solid cylindrical rod is between about 2 mm and 6 mm. For example, the length of the solid cylindrical rod is about 22 cm, and the diameter of the cylindrical rod is about 4 mm.

In various embodiments, the distal end of the solid cylindrical rod is configured to gently and repeatedly strike a bone anchor into a pilot hole formed in bone.

In various embodiments, one or more of the cylindrical tube and the solid cylindrical rod is made of a metal material. For example, the metal material may be one or more of a surgical grade stainless steel, a cobalt-chromium alloy, a titanium alloy, and the like.

In some embodiments, a bone anchor inserter includes an inserter body and a cylinder. The near end of the cylinder is coupled to the distal end of the inserter body and the distal end of the cylinder includes a retractable delivery chute. The bone anchor inserter also includes a bone anchor inserter rod which is slidingly located within the cylinder. The distal end of the bone anchor inserter rod is configured to retain a first bone anchor and a second bone anchor that are pre-loaded into the cylinder. The bone anchor inserter also includes a trigger coupled to the inserter body. The trigger is operable to retract the retractable delivery chute and deploy at least the first bone anchor.

In various embodiments, the first bone anchor and the second bone anchor are coupled by a suture and a sliding, locking knot.

In various embodiments, the distal end of the solid cylindrical rod is coupled to and terminated with a two prong fork, which is configured to straddle the first bone anchor.

In some embodiments, a bone anchor inserter includes an inserter body and a cylinder. The near end of the cylinder is coupled to the distal end of the inserter body. The bone anchor inserter also includes a bone anchor inserter rod slidingly located within the cylinder. The distal end of the bone anchor inserter rod is configured to retain a first bone anchor and a second bone anchor that are preloaded into the cylinder. The first bone anchor and the second bone anchor are coupled by a suture with a sliding knot. The bone anchor inserter also includes a trigger coupled to the inserter body. The trigger is configured to deploy at least the first bone anchor.

In various embodiments, the distal end of the cylinder comprises a retractable delivery chute configured to retract when the trigger is operated.

In various embodiments, the distal end of the solid cylindrical rod is coupled to and terminated with a two prong fork. The two prong fork is configured to straddle the first bone anchor.

Additional features and advantages of the embodiments disclosed herein will be set forth in the detailed description that follows, and in part will be clear to those skilled in the art from that description or recognized by practicing the embodiments described herein, including the detailed description which follows, the claims, as well as the appended drawings.

Both the foregoing general description and the following detailed description present embodiments intended to provide an overview or framework for understanding the nature and character of the embodiments disclosed herein. The accompanying drawings are included to provide further understanding and are incorporated into and constitute a part of this specification. The drawings illustrate various embodiments of the disclosure, and together with the description explain the principles and operations thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features and advantages of the present disclosure will be more fully described in, or rendered obvious by, the following detailed description of the preferred embodiments, which are to be considered together with the accompanying drawings, wherein like numbers refer to like parts and further, wherein:

FIG. 1 is a front perspective view of the anatomy or bodily structure of the rotator cuff region of the right shoulder, in accordance some embodiments described herein;

FIG. 2 a rotator cuff of the shoulder, in accordance with some embodiments described herein;

FIG. 3 is a side view of a bone anchor inserter for delivering bone anchors, in accordance with some embodiments described herein;

FIG. 4 is a close-up view of the tip of the bone anchor inserter of FIG. 4, in accordance with some embodiments described herein;

FIG. 5 is a sectional view of the tip of the bone anchor inserter of FIG. 4, in accordance with some embodiments described herein;

FIG. 6 is perspective view of the bone anchor inserter of FIG. 3 making a first pilot hole for a first bone anchor, in accordance with some embodiments described herein;

FIG. 7 is a perspective view of the bone anchor inserter of FIG. 3 deploying a first bone anchor, in accordance with some embodiments described herein;

FIG. 8 is a perspective view of the bone anchor inserter of FIG. 3 making a second pilot hole for a second bone anchor, in accordance with some embodiments described herein;

FIG. 9 is a perspective view of the bone anchor inserter of FIG. 3 ready for removal after deployment of the first and second bone anchors and fixation, tensioning, and locking sutures, in accordance with some embodiments described herein;

FIG. 10 is a perspective view of FIG. 9 after the bone anchor inserter has been removed from the region of the rotator cuff tear, in accordance with some embodiments described herein; and

FIG. 11a through FIG. 11h is a top view of various suture configurations that can be deployed using the medical device of FIG. 4, in accordance with some embodiments described herein.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION OF THE INVENTION

Reference will now be made in detail to the present preferred embodiment(s), examples of which is/are illustrated in the accompanying drawings. Whenever possible, the same reference numerals will be used throughout the drawings to refer to the same or like parts.

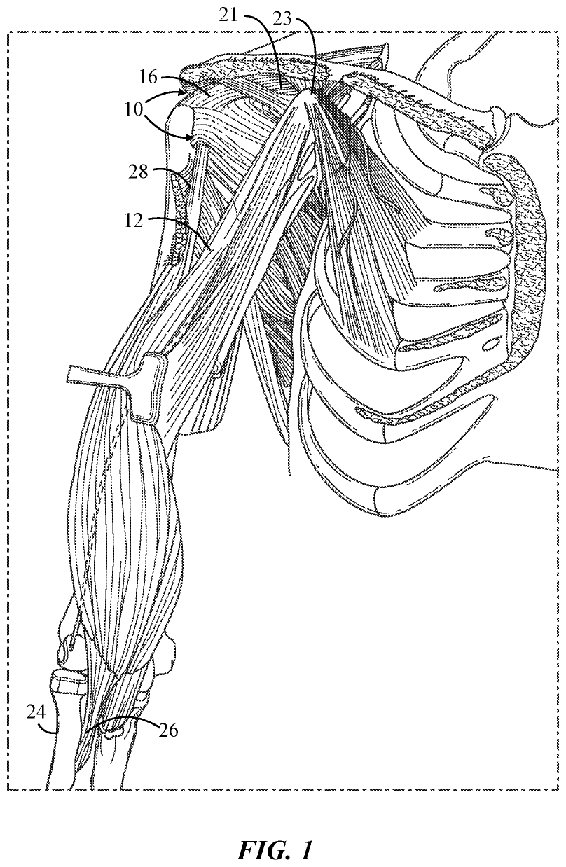

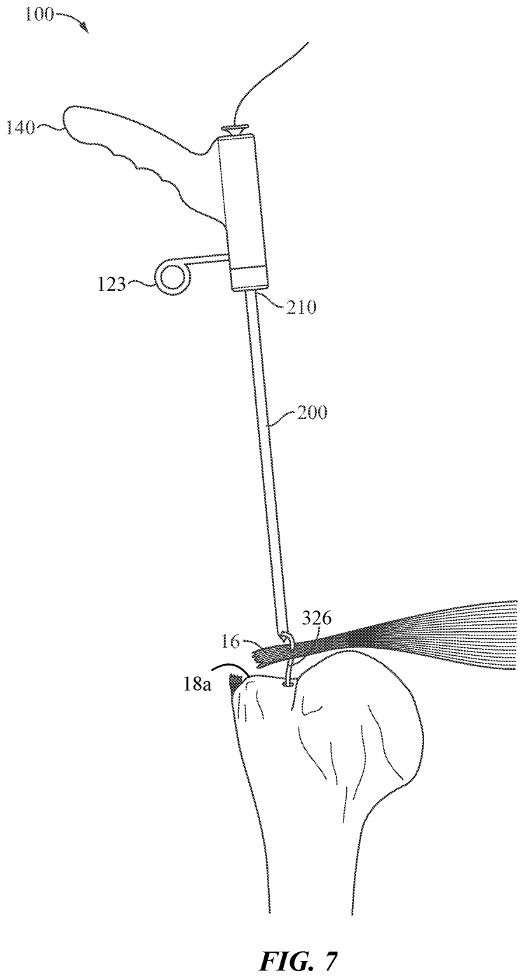

FIG. 1 shows a front perspective view some of the anatomy or bodily structure of the rotator cuff 10 region of the right shoulder. The rotator cuff 10 is a group of muscles and tendons that form a cuff over the shoulder. These muscles and tendons keep the head of the upper arm bone firmly with the shallow ball and socket joint of the shoulder and are involved in essentially all shoulder motions. The rotator cuff muscles are those muscles that surround the shoulder joint. The purpose of the rotator cuff muscles is to provide the power to lift and rotate the arm. As a person ages these muscles become thinner and are prone to rupture, sometimes with minimal trauma. In a younger patient, any rupture is usually associated with significant trauma. When the rotator cuff tendon 16 tears it usually does so from its insertion on the bone FIG. 7, 18a.

The shoulder joint falls under the category of diarthrosis joints, which are freely moveable joints. The shoulder joint is the most flexible and mobile joint in the human bone. There are three joints in the shoulder region. The acromioclavicular joint, the sternoclavicular joint, and the glenohumeral joint, the latter being the actual shoulder joint. The glenohumeral joint is attached to the rest of the skeletal system by the acromioclavicular joint and the sternoclavicular joint.

The main bone components of the glenohumeral joint are the head of the humerus 18, or bone of the upper arm, and the glenoid fossa of the scapula, glenoid labrum, or the shoulder blade bone (socket). The scapula also connects with the short head biceps tendon 12, long head bicep tendon 28, or collar bone forming the acromioclavicular joint, and the sternum or breastbone, and collectively these form the sternoclavicular joint. The distal biceps insertion 26 connects to the radius 24. While the shallow socket of the glenoid provides the shoulder joint with the most mobility of any joint in the human body it does so at the risk of the humeral head 18 slipping or being displaced during forceful movements. To prevent this, the shoulder joint is surrounded not only by the glenohumeral ligaments and capsule, but also by the large and powerful muscles of the rotator cuff 10. The rotator cuff 10 comprises four major muscles, the infraspinatus, the subscapularis, the supraspinatus, and the teres minor. The following muscles also contribute to the movement of the shoulder, the deltoid, the latissimus dorsi, the pectoralis major, and to a lesser extent the teres major.

These muscles attach to the bones in the human body via thick, fibrous connective tissue called tendons. These tendons transmit the force generated by respective muscles. It is these tendons in the shoulder joint that are sustainable to injury. A rotator cuff tendon 16 tear is a rip in the group of four muscles and tendons that stabilize the shoulder joint and enables the arms to be lifted and/or rotated. A rotator cuff tendon 16 tear may also be called a complete tear, or a full-thickness tear. Rotator cuff injuries are common and increase with age. These types of injuries may occur in athletes participating in sports like tennis, baseball, and the like, and people who have jobs that require repeated overhead activity such as house painters, and the like. These types of activities can damage the rotator cuff 10 over time. The rotator cuff can also be injured in an accident or during a fall. Millions of people suffer from rotator cuff injuries. A rotator cuff injury can cause a dull ache in the shoulder that may worsen at night. Magnetic resonance imaging can be used to reveal the existence and severity of any rotator cuff tendon 16 tears.

During a procedure, for example, the surgeon makes a number of small incisions, 2-4 in the skin proximate to the shoulder joint. An arthroscopic camera is inserted through one of the small incisions. The arthroscopic camera allows the surgeon to see inside the shoulder joint without the need for a large incision and determine the level damage to the rotator cuff tendon 16. Any surgery that can be performed with the aid of the arthroscope camera is performed at this time.

Arthroscopic surgery, also known simply as arthroscopy, is a minimally invasive orthopedic procedure for diagnosing and treating joint problems. It involves the use of a narrow tube attached to a fiber-optic video camera, an arthroscope, inserted through a tiny keyhole incision. The view inside the joint is transmitted to a high-definition video monitor. In some embodiments, specialized surgical tools access the joint through one or more tiny keyhole incisions.

In some embodiments, the surgeon can repair some small and medium size tears in good quality rotator cuff tendon 16 with very thin surgical instruments inserted through these small incisions. However, if the tear) in the rotator cuff tendon 16 is too large to be repaired with just the arthroscopic technique then a further small, 3 cm to 5 cm long, incision on the side of the shoulder may be made. Combining both the small open technique and with the arthroscopic technique is referred to as a mini open repair and provides excellent exposure of the rotator cuff tendon 16 and tear. The medical procedure may also be performed through a large (open) incision if the tear is exceptionally large or complete.

The arthroscopic camera which may also comprise lights, displays a video image of the tear in the rotator cuff tendon 16 on a monitor during the surgical procedure. Various surgical instruments are inserted through one or more of the other incisions. Loose fragments of the rotator cuff tendon 16 and the like, are removed. The surgeon may also remove bone (not shown) from the underside of the acromion 21 a bony projection of the scapula (shoulder blade) to prevent pinching the rotator cuff tendon 16. The surgeon then repairs the tear using a combination of sutures 326 and bone anchors 300. If the rotator cuff tendon 16 tear is severe then additional sutures 326 and bone anchors 300 may be required.

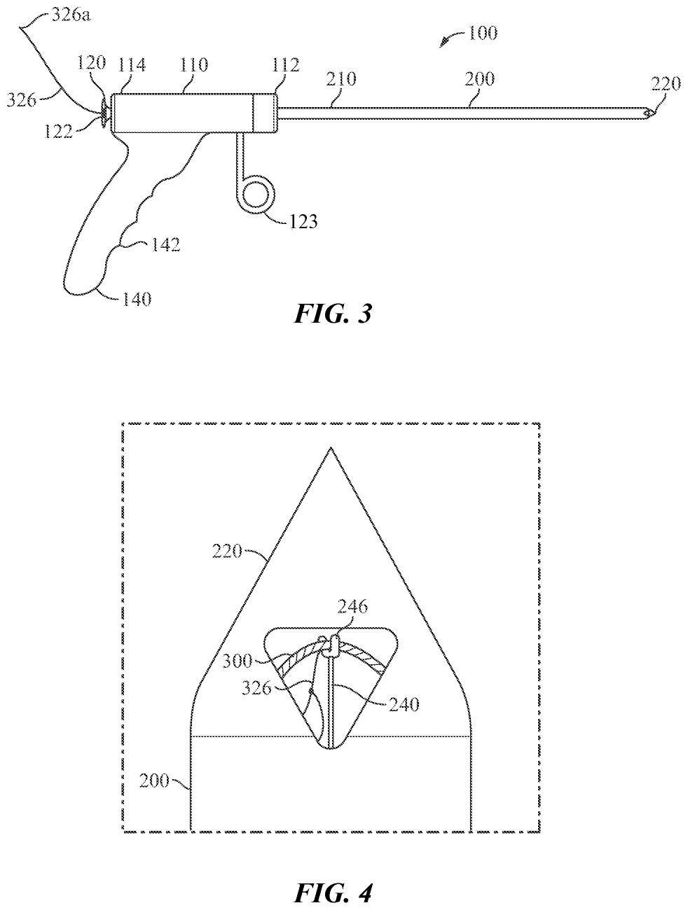

FIG. 3 shows a side view of a hand-held medical orthopedic implant device or bone anchor inserter 100 for delivering bone anchors 300 during a medical procedure through the soft tissue of a rotator cuff tendon into the upper portion of the arm bone or underlying humerus 18a. More specifically FIG. 3 shows a bone anchor inserter 100 that allows delivery of soft tissue fixation to the underlying humerus 18a at two fixation points. The two fixation points are coupled by a suture 326 comprising a sliding knot)to apply quick and easy compression along the soft tissue-bone interface between the two bone anchors 300.

In various embodiments, the bone anchor inserter 100 comprises a longitudinal body 110 and a cylindrical tube 200 having a near end 210 coupled to a distal end 112 of the longitudinal body 110. The bone anchor inserter 100 also includes a solid cylindrical rod 240 slidingly located within the cylindrical tube 200. The distal end of the solid cylindrical rod 240 is configured to retain a first bone anchor 300a and a second bone anchor 300b. The bone anchor inserter 100 also includes a top plate 120 coupled to a near end 114 of the longitudinal body and a deployment trigger 123 coupled to a middle of the underside of the longitudinal body. The deployment trigger 123 is configured to deploy the first bone anchor 300a and the second bone anchor 300b retained by the distal end of the solid cylindrical rod 240. The bone anchor inserter 100 also includes a grip 140 coupled to a rear of the underside longitudinal body 110. The grip 140 is operable to position and manipulate the distal end 210 of the cylindrical tube 200.

In some embodiments, the arthroscope camera further allows the surgeon to visualize the positioning of the distal end 220 of the cylindrical tube 200 of the bone anchor inserter 100 and insertion of the bone anchor 300 as disclosed below to repair the rotator cuff tendon 16 tear.

In various embodiments, the cylindrical tube 200 is coupled to the body of the bone anchor inserter 100. For example, the near end 210 of the cylindrical tube 200 may be configured to be pressed into and lockingly engage a recess in the body of the bone anchor inserter 100. One or more of the near end 210 of the cylindrical tube 200 and the recess within the body of the bone anchor inserter 100 may be tapered. In some embodiments, the near end 210 of the cylindrical tube 200 may be thread and be configured to lockingly engage the recess within the body of the bone anchor inserter 100 by means of a matching threaded recess.

In various embodiments, the distal end 220 of the cylindrical tube 200 is configured to make one or more pilot holes 610a. In some embodiments, the distal end 220 of the cylindrical tube 200 further comprises a retractable delivery chute.

In various embodiments, the cylindrical tube 200 is reusable and made of a durable, machine washable material that can be disinfected and sterilized. In some embodiments, the cylindrical tube 200 is disposable solution, and the cylindrical tube 200 is recycled or safely disposed of to prevent infections and cross contamination between different patients.

In various embodiments, the cylindrical tube 200 is a long hollow tube made of a metal material. For example, the cylindrical tube 200 is made of one or more of surgical grade stainless steel, cobalt-chromium alloy, titanium alloy, and the like.

In various embodiments, deployment trigger 123 is configured to deploy a bone anchor 300 into a pilot hole. In some embodiments, the deployment trigger 123 is configured to open the retractable delivery chute located at the distal end 220 of the cylindrical tube 200 prior to deployment of the bone anchor 300 into the pilot hole.

In various embodiments, the retractable delivery chute located at the distal end of the cylindrical tub 200 is configured to slide up and down the interior of the cylindrical tube 200. In some embodiments, the movement of the retractable delivery chute is responsive to the position of the deployment trigger 123. The retractable delivery chute prevents contamination and/or blocking of the distal end 220 of the cylindrical tube 200 with material from the soft tissue of the rotator cuff tendon 16.

In some embodiment, the retractable delivery chute is a hinge or flap. For example, a living hinge constructed form a soft flexible material having excellent fatigue resistance, for example nylon 12. Nylon 12 is a good general-use plastic with broad additive applications and is known for its toughness, tensile strength, impact strength and ability to flex without fracture. It is this ability to flex without fracture that makes nylon 12 suitable for the at least the retractable delivery chute portion of the cylindrical tube 200. The living hinge may also be made from acrylonitrile butadiene styrene.

The retractable delivery chute is configured to be closed when the distal end 220 of the cylindrical tube 200 is being used to pierce a hole through the soft tissue of the tendon and open when a bone anchor 300 is deployed. In some embodiments, the retractable delivery chute is a flap. The retractable delivery chute is configured to seal the distal end 220 of the of the cylindrical tube 200 when the distal end 220 is pressed against the soft tissue and the like and open when the bone anchor 300 is deployed.

In various embodiments, the body of the bone anchor inserter 100 comprises a top plate 120. The top plate 120 comprises an eyelet aperture 122 formed by a longitudinal cavity in the center of the top plate 120. The longitudinal cavity allows at least the tail 326a of the suture 326 to be feed through the eyelet aperture 122, thereby enabling the surgeon to tension and lock the suture 326 in place.

In various embodiments, the body of the bone anchor inserter 100 comprises a handle or grip 140. The grip 140 is formed of a material, and has a shape, which is comfortable to firmly gripped and manipulated. The grip 140 may be formed, or at least coated, in a material having a high coefficient of friction. For example, having a coefficient of friction of about 0.5 or greater. In some embodiments, the material is Polyvinyl (PVC). PVC is a synthetic thermoplastic composed of vinyl chloride. PVC offers high-grip control and is resistant against chemicals, oils, greases, and other substances. In various embodiments, at least the grip 140 of the bone anchor inserter 100 comprises finger grooves configured to make the grip 140 easier to grip and manipulate.

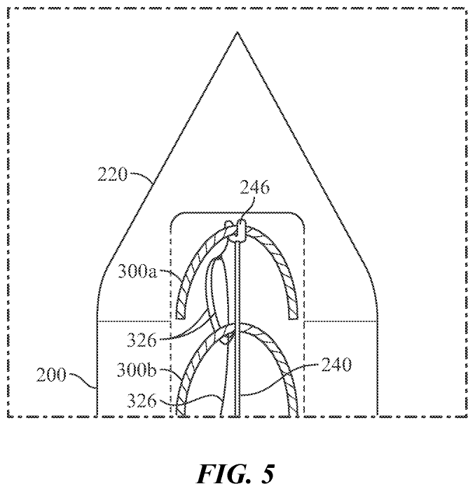

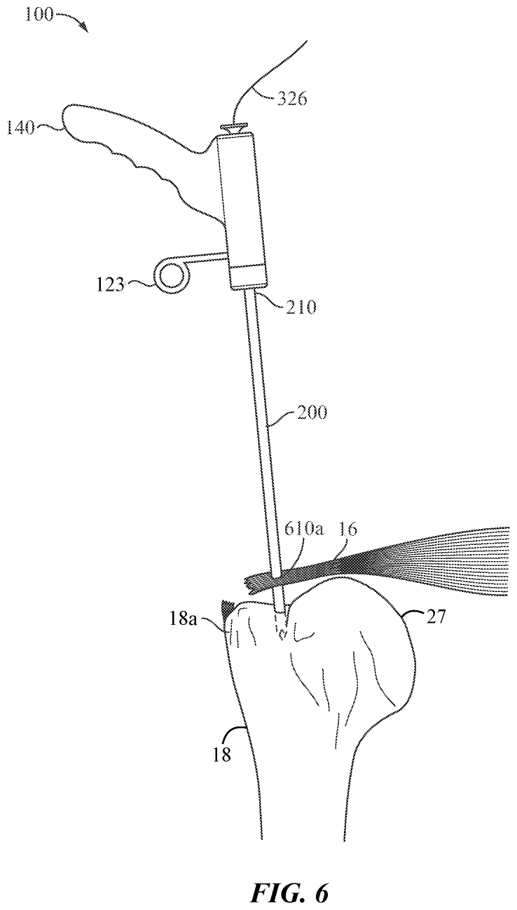

FIG. 4 shows a close-up view of the distal end 220 of the cylindrical tube 200 of the bone anchor inserter 100 of FIG. 3 with the retractable window of the delivery chute in the open position and a bone anchor 300 ready for deployment. Also shown in FIG. 5 is a solid cylindrical rod 240, more specifically a distal end 246 of the solid cylindrical rod 240. In various embodiments, the distal end 246 of the solid cylindrical rod 240 is coupled to and terminated with a two prong fork 246. The two prong fork at the distal end 246 is configured to retain and support at least one bone anchor 300 prior to deployment.

In various embodiments, the length of the solid cylindrical rod 240 is between about 15 cm and about 25 cm and the diameter the solid cylindrical rod 240 is between about 2 mm and 6 mm. For example, the length of the solid cylindrical rod 240 is about 22 cm, and the diameter of the cylindrical rod is about 4 mm.

FIG. 5 shows a close-up sectional view of the distal end 220 of the cylindrical tube 220 of the anchor inserter 100 of FIG. 4. Also shown in FIG. 5 is a first bone anchor 300a supported by the two prong fork 246 of the solid cylinder rod 240 and a second bone anchor 300b ready for deployment. The two prong fork 246 allows delivery of the first bone anchor 300a and the second bone anchor 300b, while also delivering a pre-loaded sliding and locking knot to apply quick and easy compression along the soft tissue-bone interface between, such that the soft tissue of the rotator cuff tendon 16 is secured to the humerus. The suture 326 comprising the sliding knot is attached to both the first bone anchor 300a and the second bone anchor 300b Also shown in FIG. 5 is the locking suture 326 feeding out towards the body of the anchor inserter 100 by way of the cylindrical tube 200.

FIG. 7 shows a perspective view of the bone anchor inserter 100 of making a first pilot hole to accept the suture 326 in the medial row for the first bone anchor.

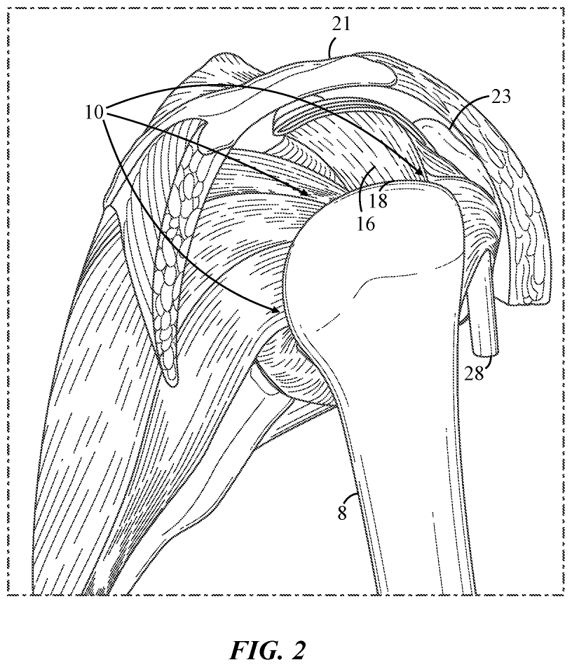

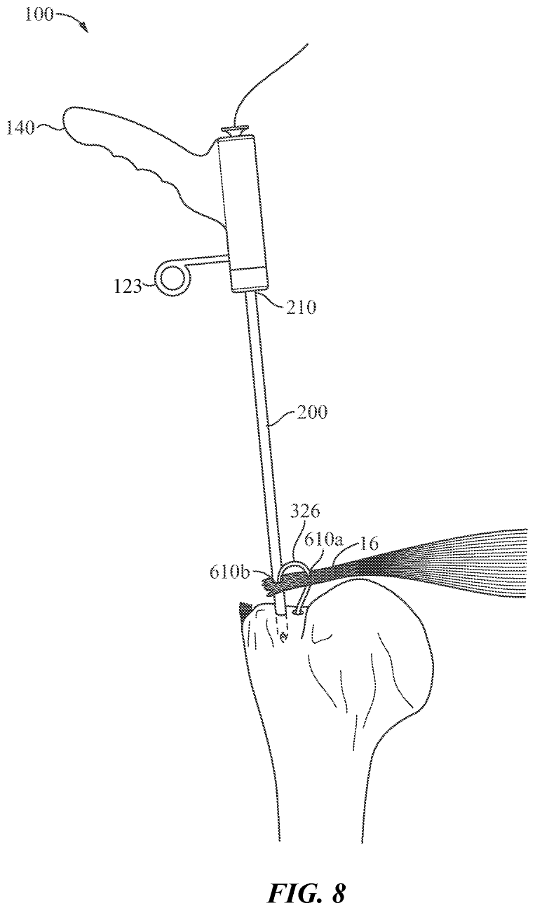

FIG. 8 shows a perspective view of the bone anchor inserter 100 of with the first bone anchor 300a deployed. After deployment of the first bone anchor 300a through the tendon and into bone, the distal end 220 of the cylindrical tube 200 of the bone anchor inserter 100 remains within the region of the subacromial space as shown. The subacromial space refers to the space above the shoulder's glenohumeral joint (ball-and-socket joint) and below the acromion 21 and coracoid 23 (See FIG. 2), the top-most bone of the shoulder. Soft tissues, such as the bicep tendon, rotator cuff tendon 16, and bursa are located within the subacromial space.

FIG. 8 shows the bone anchor inserter 100 of making a second pilot hole 610b, the lateral row fixation, for a second bone anchor 300b in preparation for the suture 326 comprising the sliding knot bridging the gap between the medial row fixation formed by the first bone anchor 300a, and the lateral row fixation formed by the second bone anchor 300b through tendon and into bone, along the end portion of the rotator cuff tendon 16.

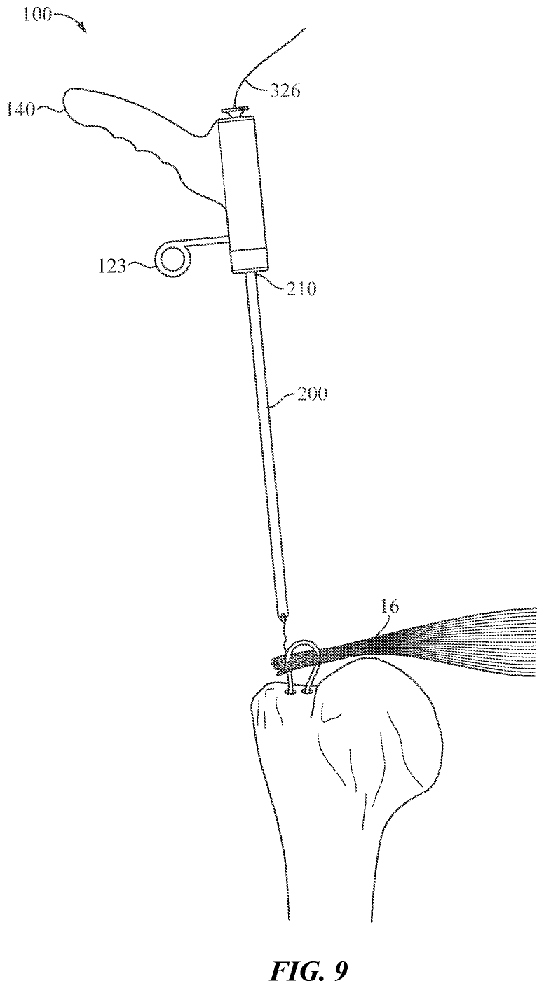

FIG. 9 shows the bone anchor inserter 100 of ready for removal after making the pilot hole 610b and deployment of the second bone anchor 300b. In FIG. 9 the suture 326 comprising the sliding knot bridges and locks the gap between the medial row fixation, formed by the first bone anchor 300a and the lateral row fixation formed by the second bone anchor 300b, along the end portion of the soft tissue of the rotator cuff tendon 16.

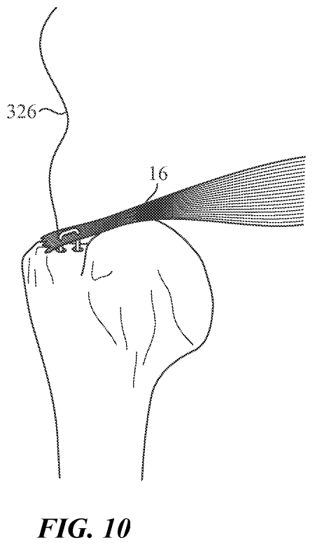

FIG. 10 is another view of FIG. 9 after the bone anchor inserter has been removed. In FIG. 10 the sliding knot has slid down and locked the fixation formed by the medial row fixation formed by the first bone anchor and the lateral row fixation formed by the second bone anchor 300b. Specifically, the sliding knot has slide down the suture 326 and locked the fixation obtained at both the medial anchor point, formed by the first bone anchor, and the lateral anchor point, formed by the second bone anchor, into the humerus.a As a result, compression is now being applied to the region bridging these two anchor points by the suture and sliding knot and rotator cuff tendon 16.

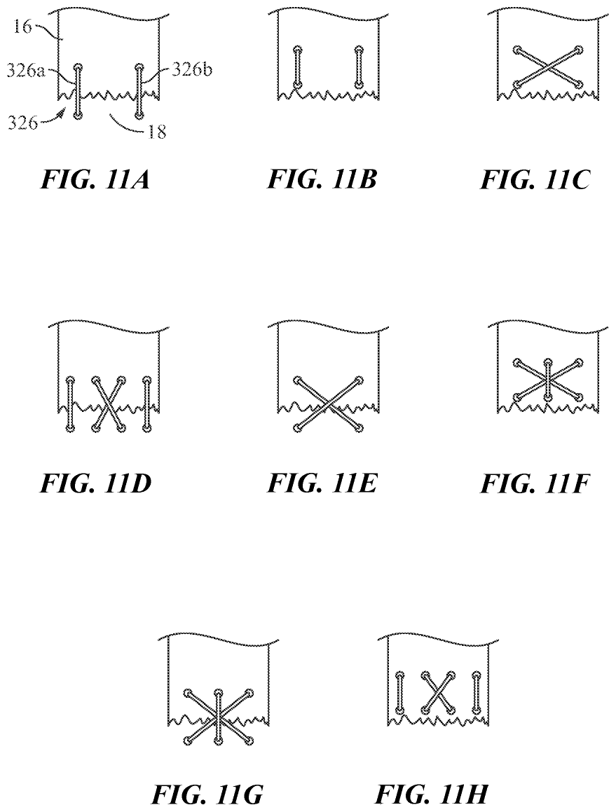

FIG. 11a through FIG. 11h are top views of various configurations of deployed sutures 326, bridging two or more bone anchors 300, that can be obtained by preloading the bone anchor inserter 100 with two or more bone anchors 300 linked by different configurations of sutures 326. For the sake of clarity, the bone anchor(s) 300 have been omitted. While FIG. 11 shows various configurations of sutures 326 and the anchor points for two or more bone anchors 300 that can be deployed using the bone anchor inserter 100 other configurations are contemplated as well.

FIG. 11a shows a first configuration of bridged anchor points that are locked and compressed by sutures 326. In FIG. 11a two of the anchor points are situated in the upper portion of the arm bone or humerus 18a and two of the anchor points are situated humerus 18a after passing through the soft tissue of the rotator cuff tendon 16. The first suture 326a and the second suture 326b are parallel to each other.

FIG. 11b shows a second configuration of bridged anchor points that are locked and compressed by sutures 326. In FIG. 11b all the anchor points are situated in the humerus 18a after passing through the soft tissue of the rotator cuff tendon 16. The first suture 326a and the second suture 326b are parallel to each other.

FIG. 11c shows a third configuration of bridged anchor points that are locked and compressed by sutures 326. In FIG. 11c all the anchor points are situated in the humerus 18a after passing through the soft tissue of the rotator cuff tendon 16. The first suture 326a and the second suture 326b are crossed.

FIG. 11d shows a fourth configuration of bridged anchor points that are locked and compressed by sutures 326. In FIG. 11d all the anchor points are situated in the humerus 18a after passing through the soft tissue of the rotator cuff tendon 16. An inner pair of sutures 326 are crossed and are located between an outer pair of sutures that are parallel to each other.

FIG. 11e shows a fifth configuration of bridged anchor points that are locked and compressed by sutures 326. In FIG. 11e two of the anchor points are situated in the humerus 18 after passing through the soft tissue of the rotator cuff tendon 16 and two of the anchor points are situated in only the humerus 18. The first suture 326a and the second suture 326b are crossed.

FIG. 11f shows a sixth configuration of bridged anchor points that are locked and compressed by sutures 326. In FIG. 11f all the anchor points are situated in the humerus 18a after passing through the soft tissue of the rotator cuff tendon 16 and the three sutures 326a, 326b, and 326c are crossed.

FIG. 11g shows a seventh configuration of bridged anchor points that are locked and compressed by sutures 326. In FIG. 11g three of the anchor points are situated in the humerus 18a after passing through the soft tissue of the rotator cuff tendon 16 and three of the anchor points are situated in only the humerus 18a, The three sutures 326a, 326b, and 326c are crossed with each of the sutures 326a, 326b, and 326c having one anchor point situated in the humerus 18a after passing through the soft tissue of the rotator cuff tendon 16 and another anchor point situated in only the upper portion of the arm bone or humerus 18.

FIG. 11h shows a seventh configuration of bridged anchor points that are locked and compressed by sutures 326. In FIG. 11h eight of the anchor points are situated in the humerus 18a after passing through the soft tissue of the rotator cuff tendon 16 and two of the anchor points are situated in only the humerus 18a. An inner pair of sutures 326 are crossed and are located between a pair of sutures 326 that are parallel to each other, which are in turn located between an outer pair of sutures 326 having one anchor point situated in the humerus 18a after passing through the soft tissue of the rotator cuff tendon 16 and another anchor points situated in only the humerus 18.

In various embodiments, the surgical procedure also comprises making a second accessory portal incision, where the second incision is used to introduce a cannula (not shown). In some embodiments, the accessory portal incision is located directly over the tear 16 in the soft tissue of the rotator cuff tendon 16 In various embodiments, the surgeon relies entirely on the primary viewing portal to view the procedure.

In some embodiments, a cannula (not shown) is placed into the accessory portal incision prior to forming the one or more pilot holes 610. The distal end of the cannulated is manipulated with the aid of an arthroscopic camera until the distal end of the cannula proximate to the tear 16. The diameter of the cannula is between about 6 mm and about 10 mm. In some applications, the diameter of the cannula is between about 7 mm and about 8.5 mm. The body of the cannula is made from a transparent plastic material. The cylindrical tube 200 slides down the interior of the cannula until the distal end 220 of the cylindrical tube 200 is proximate to the primary bone attachment site or designated primary fixation site.

In various embodiments, the distal end 220 of the cylindrical tube 200 is then placed over the tear 16 at the designated primary fixation site, which is usually located proximate to the junction between the tendon and the muscle and tapped into the bone of the humerus to form the one or more bone anchor pilot holes 610.

In various embodiment, the distal end 220 of the cylindrical tube 200 is used to make one or more pilot holes 610 in the humerus 18a for the bone anchor 300. In some embodiments, the depth of each of the one or more pilot holes 610 is about 15 mm and the spacing between the one or more pilot holes 610 is between about 4 mm and about 10 mm. In some embodiments, the spacing between the one or more pilot holes 610 is configured to match the width of the prongs of the bone anchor 300.

In various embodiments, the soft tissue of the rotator cuff tendon 16 is positioned over the designated primary fixation site. The first anchor 300a is tapped through the soft tissue and into the upper portion of the arm bone or humerus 18a. In some embodiments, the bone anchors 300 are made from a special biodegradable plastic that the body will naturally absorb over time. The bone anchor inserter 100 is then withdrawn just outside the original soft tissue site and then positioned at the designated secondary fixation site. The bone anchor inserter 100 is impacted through into the upper portion of the arm bone or humerus 18a, and in some embodiments the soft tissue of the tendon, and the second anchor 300b is deployed. The bone anchor inserter 100 is completely withdrawn, and the sliding locking suture 326 is tensioned, applying compression of the soft tissue to bone at the two anchor fixation points and across the two points of fixation. The bone anchor inserter 100 may also be used to secure a suture 326 across the span of a ligament from bone to bone to provide “protection” to the ligament repair/reconstruction.

One advantage of the proposed bone anchor inserter 100 is that the bone anchors 300 are configured to be self-tapping.

Another advantage of the proposed anchor inserter 100 is that the suture 326 can be pre-loaded and pre-tied between the bone anchors 300 thereby allowing the complete assembly to be deployed without having to manually assemble and link the bone anchors 300 with a suture 326 after deployment.

Yet another advantage of the proposed bone anchor inserter 100 is that the suture 326 is adjustable and self-locking thereby avoiding the need to tie knots or finalize the tension with a further implants or bone anchors 300.

Yet another advantage of the proposed bone anchor inserter 100 is that the sutures 326 are pre-loaded and pre-tied between the bone anchors 300. This configuration has the advantage of not requiring the either the surgeon or the surgical assistant to manually assemble and link the bone anchors 300 with a tensioning suture 326 and locking knot during the medical procedure.

Yet another advantage of the proposed bone anchor inserter 100 is that the tensioning suture 326 is adjustable and self-locking by way of the locking knot 328. This configuration avoids the need to tie knots or finalize the tension with further implants or bone anchors 300, sutures 326, and locking knots 328.

In various embodiments, the diameter of the solid cylindrical rod 240 is significant smaller than the width of the bone anchor 300. In some embodiments, the diameter of solid cylindrical rod 240 between about 2 mm and about 8 mm. For example, the diameter of solid cylindrical rod 240 is equal to or less than about 4 mm. In some embodiments, the distal end of cylindrical rod 240 is configured to lockingly engage the middle of the bone anchor 300. For example, the distal end of the cylindrical rod 240 is coupled to, and terminated with, a two prong fork. The two prong fork 246 is configured to straddle and support the first bone anchor 300.

While the bone anchor inserter 100 is described in the context of repairing a tear in the rotator cuff tendon 16 of the shoulder other types of tendons are contemplated as well.

While preferred embodiments of the present invention have been shown and described herein, it will be obvious to those skilled in the art that such embodiments are provided by way of example only. Numerous variations, changes, and substitutions will now occur to those skilled in the art without departing from the invention, in accordance with the claims. It should be understood that various alternatives to the embodiments of the invention described herein may be employed in practicing the invention.

What is claimed is:

1. A bone anchor inserter apparatus, the bone anchor inserter comprising:
a longitudinal body;
a cylindrical tube having a proximal end coupled to a distal end of the longitudinal body;
a solid cylindrical rod slidingly located within the cylindrical tube, wherein a distal end of the solid cylindrical rod is operable to retain a first bone anchor and a second bone anchor;
a top plate coupled to a proximal end of the longitudinal body;
a deployment trigger coupled to an underside of the longitudinal body, wherein the deployment trigger is operable to deploy the first bone anchor and the second bone anchor retained by the distal end of the solid cylindrical rod; and
a grip coupled to an underside of the longitudinal body,
wherein the grip is located proximal to the deployment trigger, and
wherein the grip is operable to position and manipulate the distal end of the cylindrical tube.

2. The bone anchor inserter apparatus of claim 1, wherein the cylindrical tube is pre-loaded with the first bone anchor and the second bone anchor; and wherein the first bone anchor and the second bone anchor are coupled by a suture comprising a sliding knot.

3. The bone anchor inserter apparatus of claim 2, wherein the top plate comprises an eyelet aperture formed by a longitudinal cavity in a center of the top plate, and
wherein the longitudinal cavity in the center of top plate is operable to allow at least a tail of the suture to be feed through the eyelet aperture.

4. The bone anchor inserter apparatus of claim 3, wherein the tail of the suture and the sliding knot are operable to apply a compression force along a soft-tissue bone interface between the first bone anchor and the second bone anchor.

5. The bone anchor inserter apparatus of claim 3, wherein a distal end of the cylindrical tube is in fluid communication with the eyelet aperture formed by the longitudinal cavity in the center of the top plate.

6. The bone anchor inserter apparatus of claim 1, wherein a distal end of the solid cylindrical rod is coupled to and terminates with a two prong fork, and
wherein the two prong fork is operable to straddle and support the first bone anchor.

7. The bone anchor inserter apparatus of claim 6, wherein a length of the solid cylindrical rod is between 15 cm and 25 cm,
wherein the length of the solid cylindrical rod and the two prong fork is 22 cm,
wherein a diameter of the solid cylindrical rod is between 2 mm and 6 mm, and
wherein the diameter of the solid cylindrical rod is about 4 mm.

8. The bone anchor inserter apparatus of claim 1, wherein the proximal end of the cylindrical tube is operable to lockingly engage the distal end of the longitudinal body, and
wherein the proximal end of the cylindrical tube is threaded and operable to engage a matching threaded recess in the distal end of the longitudinal body.

9. The bone anchor inserter apparatus of claim 1, wherein a length of the cylindrical tube is between 15 cm and 25 cm.

10. The bone anchor inserter apparatus of claim 1, wherein a distal end of the cylindrical tube tapers to a sharp point that operable to make at least one pilot hole for the first bone anchor and the second bone anchor,
wherein a length of the sharp point is between 10 mm and 20 mm.

11. The bone anchor inserter apparatus of claim 1, wherein the distal end of the cylindrical tube comprises a retractable delivery chute, and
wherein the retractable delivery chute is operable to deliver the first bone anchor and the second bone anchor.

12. The bone anchor inserter apparatus of claim 1, wherein a distal end of the solid cylindrical rod is operable to be used to repeatedly strike the first bone anchor and the second bone anchor.

13. The bone anchor inserter apparatus of claim 1, wherein one or more of the cylindrical tube and the solid cylindrical rod comprises a metal material, and
wherein the metal material is one or more of a surgical grade stainless steel, a cobalt-chromium alloy, and a titanium alloy.

14. The bone anchor inserter of apparatus claim 1, wherein one or more of the cylindrical tube and the solid cylindrical rod is made of metal.

15. A bone anchor inserter system, comprising:
an inserter body;
a cylinder comprising:
a proximal end coupled to a distal end of the inserter body; and
a retractable delivery chute at a distal end;
a bone anchor inserter rod slidingly located within the cylinder, comprising:
a distal end, operable to retain a first bone anchor and a second bone anchor; and
a trigger, operable to retract the retractable delivery chute and deploy at least the first bone anchor, coupled to the inserter body,
wherein the first bone anchor and the second bone anchor are pre-loaded into the cylinder.

16. The bone anchor inserter system of claim 15, wherein a distal end of the solid cylindrical rod comprises a two prong fork that is operable to support at least the first bone anchor.

17. The bone anchor inserter system of claim 15, wherein the first bone anchor and the second bone anchor are slidingly coupled by a suture.

18. A bone anchor inserter apparatus, the bone anchor inserter comprising:
an inserter body;
a cylinder having a proximal end coupled to a distal end of the inserter body;
a bone anchor inserter rod slidingly located within the cylinder, wherein a distal end of the bone anchor inserter rod is operable to retain a preloaded first bone anchor and a second bone anchor; and
a trigger coupled to the inserter body, wherein the trigger is operable deploy at least the first bone anchor,
wherein the first bone anchor and the second bone anchor are coupled by a suture and a sliding knot.

19. The bone anchor inserter apparatus of claim 18, wherein a distal end of the cylinder comprises a retractable delivery chute, and
wherein the trigger is operable to retract the retractable delivery chute as the at least the first bone anchor is deployed.

20. The bone anchor inserter apparatus of claim 18, wherein a distal end of the solid cylindrical rod is coupled to a two prong fork that is operable to straddle a middle of the first bone anchor, and
wherein the first bone anchor and the second bone anchor are coupled by a suture comprising a slidingly knot.
